(19) United States
(12) Plant Patent Application Publication
(10) Pub. No.: US 2004/0128726 P1
(43) Pub. Date: Jul. 1, 2004

(54) Nemesia plant named 'Balardeblu'

(75) Inventor: Scott C. Trees, Arroyo Grande, CA (US)

Correspondence Address: WOOD, PHILLIPS, KATZ, CLARK & MORTIMER, 500 W. MADISON STREET, SUITE 3800, CHICAGO, IL, 60661, US

(73) Assignee: Ball FloraPlant, a division of Ball Horticultural Company

(21) Appl. No.: 10/329,732

(22) Filed: Dec. 26, 2002

(51) Int. Cl. A01H 5/00

(52) U.S. Cl. PLT/263

(57) ABSTRACT

A new and distinct Nemesia plant named ‘Balardeblu’, characterized by its dark violet colored flowers, leaves of a medium green color and upright growth habit.

### LATIN NAME OF THE GENUS AND SPECIES OF PLANT CLAIMED

Nemesia×hybrida

### VARIETY DENOMINATION

‘Balardeblu’

### BACKGROUND OF THE INVENTION

The present invention comprises a new and distinct Nemesia plant, botanically known as Nemesia×hybrida, and hereinafter referred to by the name ‘Balardeblu’.

‘Balardeblu’ is the result of open pollination, with the female parent being ‘Blue Bird’ (not patented). The new cultivar was discovered by the inventor during October 2000, in a controlled environment at Arroyo Grande, Calif.

Asexual reproduction of the new cultivar has been carried out at Arroyo Grande, Calif. and West Chicago, Ill. by terminal tip cuttings and has demonstrated that the characteristics of the new cultivar as herein described are firmly fixed and retained through successive generations of such asexual propagation.

### SUMMARY OF THE INVENTION

It was found that the cultivar of the present invention:

(a) exhibits flowers of a dark violet color,

(b) forms medium green foliage,

(c) has a good basal branching character, and

(d) exhibits an upright growth habit.

The new cultivar of the present invention can be compared to its female parent, ‘Blue Bird’. In side-by-side comparisons, plants of the new cultivar exhibit shorter branches with fewer leaves per branch, and darker flower color than ‘Blue Bird’.

### BRIEF DESCRIPTION OF PHOTOGRAPH

The accompanying photographs show as nearly true as it is reasonably possible to make the same in color illustrations of this type, typical flower and foliage characteristics of the new cultivar. Colors are as accurately represented as can be made by conventional photography. The plants were grown in 10 cm pots for 9 weeks in a greenhouse at West Chicago, Ill.

FIG. 1 illustrates the overall growth habit of the new cultivar.

FIG. 2 illustrates a close-up view of individual flowers of the new cultivar.

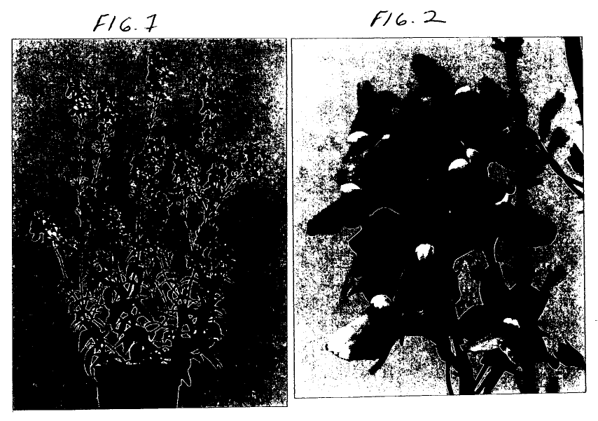

### DETAILED BOTANICAL DESCRIPTION

‘Balardeblu’ has not been observed under all possible environmental conditions to date. Accordingly, it is possible that the phenotype may vary somewhat with variations in the environment, such as temperature, light intensity, and day length. The chart used in the identification of colors described herein is the R.H.S. Colour Chart of The Royal Horticultural Society, London, England. The color values were determined on Apr. 22, 2002. The readings were taken between 1:00 and 3:00 p.m. under natural daylight conditions. The plants were produced from cuttings taken from stock plants and were grown in a double polycarbonate covered greenhouse under conditions comparable to those used in commercial practice. The plants were grown utilizing a soilless growth medium and maintaining temperatures of approximately 65° to 75° F. (18° to 24° C.) during the day and approximately 50° to 60° F. (10° to 15° C.) during the night and light levels of 4,000 to 7,000 footcandles. Plants used for the following descriptions and measurements were grown in 10 cm pots for 9 weeks from rooted cuttings.

Classification:

Botanical.—Nemesia×hybrida, cultivar ‘Balardeblu’.

Parentage:

Female parent.—Commercially available ‘Blue Bird’.

Propagation:

Type cutting.—Terminal tip.

Time to initiate roots.—Approximately 7 to 10 days.

Time to develop roots.—Approximately 14 to 21 days.

Root description.—Fibrous, branching.

Plant description:

Habit of growth.—Moderately vigorous with good branching. Pinching improves branching. A mature plant, 10 weeks after the planting of a rooted cutting, commonly measures approximately 38.6 cm in height and approximately 62 cm in diameter with an average of 5.4 primary branches.

Form.—Upright.

Primary branch.—Length: Approximately 9.9 cm. Diameter: Approximately 2 mm. Texture: Moderately pubescent. Color: 146A. Internode length at middle of branch: Approximately 2.2 cm.

Foliage.—Leaves are non-fragrant, single, opposite, at an acute angle to the stem. Leaves are narrow ovate with serrate margin, acute apex and sessile base. Upper and lower surfaces are glabrous with no pubescence. Leaf length: Approximately 3.6 cm. Leaf width: Approximately 1.7 cm. Color: Upper surface of mature foliage is 137A, lower surface of mature foliage is 144B. Both upper and lower surfaces have palmate venation closest to 144B.

Flowering description:

Flowering habit.—Freely flowering.

Natural flowering season.—Year round in greenhouse environment and spring through autumn in outdoor garden.

Flower type.—Bilabiate, in terminal clusters. Flowers are persistent.

Peduncle.—Square, strong, with moderate pubescence, at an acute angle to the stem, approximately 5.5 cm in length and 1 mm in diameter. Peduncle color is 144B.

Bud.—Round, approximately 6 mm in length and 5 mm in diameter. Bud color is N79C.

Flower description.—Flowers are bilabiate. The upper lip has two central linear lobes with two outer obovate lobes — one on each side. All lobes have rounded tips and attenuate bases. The lower lip has two obovate lobes with rounded tips and attenuate bases. When first open, the upper surface of all lobes is 86B with palate of 5A. Upper surface of mature lobes is 86C. Upper surface of all lobes fades to N88C with age. Lower surface of all lobes, either when first opening or at maturity, is 86D.

Calyx.—Five sepals fused at base. Sepal shape: Elliptic. Sepal length: Approximately 1 mm. Sepal width: Less than 1 mm. Margin: Entire. Apex: Acute. The both surfaces are densely pubescent and 144B.

Reproductive organs.—Androecium: There are 4 stamens-2 are 8 mm in length, 2 are 6 mm in length. Anthers are coiled and 1 mm in length. Pollen is abundant and color is 11B. Gynoecium: One pistil, 2 mm in length. Stigma shape is spatulate, less than 1 mm in length and 150C. Style length is less than 1 mm and color is 150D. Ovary diameter is 1 mm and color is N144C.

Seed production: Seed production has not been observed.

Disease resistance: Resistance to pathogens has not been observed.

Hardiness zone: ‘Balardeblu’ is hardy in zones nine (9) and above.

I claim:

1. A new and distinct Nemesia plant named ‘Balardeblu’ substantially as herein shown and described, which:
(a) exhibits flowers of a dark violet color,
(b) forms foliage of a medium green color,
(c) has a good basal branching character, and
(d) exhibits an upright growth habit.
